(19) United States
(12) Patent Application Publication
(10) Pub. No.: US 2022/0160673 A1
(43) Pub. Date: May 26, 2022

(54) METHOD PREPARING SILVER-NANOCURCUMIN MATERIAL FOR INHIBITING NEW CORONAVIRUS

(71) Applicant: FULLFAITH INTERNATIONAL HOLDING LIMITED, Hong Kong (HK)

(72) Inventor: Chih-Chang Lai, Hong Kong (HK)

(21) Appl. No.: 17/103,510

(22) Filed: Nov. 24, 2020

(51) Int. Cl. A61K 31/28

(52) U.S. Cl. CPC A61K 31/28

(57) ABSTRACT

The present disclosure provides a method preparing Silver-nanocurcumin material for inhibiting new coronavirus. The turmeric powder is heated and stirred and then mixed with silver nitrate solution. After multiple steps of cooling, stirring for reaction and centrifuging and other steps, crude extract of Silver-nanocurcumin is obtained. Then, after further processes such as the multiple washing and centrifuging, a purified composition of Silver-nanocurcumin is obtained. The composition of Silver-nanocurcumin can be used to inhibit the new coronavirus and its similar viruses, and as a source of drugs for the prevention and treatment of diseases derived from the new coronavirus COVID-19 in the future.

### BACKGROUND OF THE INVENTION

### Field of the Invention

The method preparing Silver-nanocurcumin material for inhibiting new coronavirus of the present disclosure relates to a technology field about preparing Silver-nanocurcumin material that can be used to inhibit viruses and as a future prevention and treatment of new coronavirus COVID-19-derived disease.

### Description of the Prior Art

According to information obtained from Google, the new coronavirus (COVID-19), which has recently become disillusioned and highly contagious, has caused 37.3 million confirmed cases worldwide, up to 1.07 million as died of illness of Oct. 11, 2020, and the number is still rising rapidly.

Coronavirus (CoV) is a group of RNA viruses with a mantle (envelope). It is named because the crown-like protrusions can be seen under an electron microscope. Coronavirus has become the cause of severe acute respiratory diseases, from the severe acute respiratory syndrome coronavirus (SARS-CoV) in 2003, the Middle East respiratory syndrome coronavirus (MERS-CoV) in 2013, to the current serious harm to the world in 2019 the pandemic of SARS-CoV-2, the new coronavirus COVID-19 was named by the World Health Organization. Due to the high mutation rate of RNA viruses, this phenomenon helps RNA viruses to rapidly develop drug resistance and make vaccines ineffective. AIDS is an RNA virus. It is a well-known human disease. So far, there is no vaccine to treat AIDS because of the high mutation characteristics of RNA viruses.

Based on long-term research on the application of nano-silver and turmeric, the inventor found that the combination of nano-silver and turmeric can inhibit the virus.

Nano-silver has antibacterial effects, antifungal and antiviral properties and silver compounds have also been extensively studied. Nano-silver can be used in fabrics, wound dressings and medical devices. Nano-silver antibacterial agents have shown effectiveness against both Gram-positive and Gram-negative bacteria. There have been literature records that nano-silver has proven its antibacterial effect on bacteria through in vitro experiments, such as the drug-resistant methicillin-resistant Staphylococcus aureus (MRSA), Escherichia coli, Pseudomonas aeruginosa, Vibrio cholerae and Bacillus subtilis all have good antibacterial effects. Nano-silver also has good antibacterial effect on fungi such as Aspergillus niger and Candida albicans. The same nano-silver also exhibits its excellent antiviral effects against various viruses, such as HIV (Human immunodeficiency virus), Respiratory syncytial virus (RSV), Tacaribe virus (TCRV), Herpes simplex virus type 1 (herpes simplex virus-1, HSV-1), Monkeypox virus and hepatitis B virus (HBV). Therefore, the inhibition and killing of bacteria and viruses by nano-silver involves multiple mechanisms of broad effectiveness. It can destroy the virus mantle or the activity of DNA, RNA and protein of virus, to kill the virus. Even if the virus mutates, it does not affect the virus-killing properties of nano-silver. In order for the nano-silver to interact with the virus effectively, the particle size of nano-silver needs to be significantly smaller than that of the virus, for example, less than 10 nm, or preferably less than 10 nm, or preferably in the range of 3 nm-7 nm. As long as the virus is fully covered by the nano-silver particles, the function of the virus will be disturbed.

As for turmeric (Curcuma longa L. as scientific name, Turmeric as English name), the active ingredient curcumin (molecular formula C21H20O6) is a compound composed of carbon (C), hydrogen (H) and oxygen (O). Curcumin has been approved by the U.S. Food and Drug Administration (FDA), and more than 300 clinical trials have reported curcumin's beneficial protective effects on various diseases, including inflammatory diseases, neurological diseases, cardiovascular diseases, lung diseases, Metabolic disease, liver disease and cancer. A large number of research literature show that turmeric and curcumin can resist a variety of viruses, such as HIV (Human immunodeficiency virus, HIV), influenza virus, herpes simplex virus type 1 and herpes simplex virus type 2 (HSV-1 and HSV-2), Coxsackievirus, Hepatitis B Virus (HBV), Hepatitis C virus (HCV), Human Papilloma Virus, HPV), Japanese Encephalitis Virus (JEV) and Human T-lymphotropic virus 1 (HTLV-1). Therefore, turmeric and curcumin have a lot of evidence that they have inhibit various viral infections.

The inventors used nano-silver and turmeric for preparation for applying to inhibit the new coronavirus and similar viruses, and as a source of drugs for the prevention and treatment of diseases derived from the new coronavirus COVID-19 in the future.

### SUMMARY OF INVENTION

The main purpose of the present disclosure which provides a method preparing Silver-nanocurcumin material for inhibiting new coronavirus is to improve and alleviate the impact of the new coronavirus COVID-19 on global human health and economy, and break through the current human response to the new coronavirus and the bottleneck for disease prevention and treatment, using biological nanomaterials with turmeric as the mainstay and nano-silver as the supplement. According to the present disclosure, curcumin and nano-silver can be simultaneously obtained to inhibit the virus, and the problem of curcumin being unstable and not easily soluble in water and difficult to be absorbed by the human body can be solved. At the same time, the biological irritation caused by using nano-silver alone can be avoided. As above, what the method of the present disclosure is produced can be a source of drugs for the prevention and treatment of diseases derived from the new coronavirus in the future, and to provide an effective and safe drug to inhibit the new coronavirus COVID-19 to make up for the current high mutation rate of the new coronavirus that may lead to future vaccines or other relevant treatment plans not completely dealing with the solution of the new coronavirus epidemic, thereby improving the global human health and economic problems caused by the new coronavirus epidemic.

To achieve the above objective, the specific preparation method of the present disclosure includes the following steps in sequence:

step1: mixing turmeric powder with deionized water;

step2: heating to boiling;

step3: cooling and stirring;

step4: mixing the silver nitrate solution;

step5: cooling and stirring for reaction;

step6: centrifuging;

step7: taking out the supernatant;

step8: centrifuging the supernatant;

step9: collecting the precipitate to obtain crude extract of Silver-nanocurcumin;

step10: adding deionized water to mix and wash by shaking at high speed

step11: centrifuging;

step12: collecting the precipitate;

step13: repeating washing and centrifuging; and

step14: obtaining a purified composition of Silver-nanocurcumin.

In one preferred embodiment, in the step 1, 5 grams of the turmeric powder are weighed out and poured into 500 ml of the deionized water.

In one preferred embodiment, in the step 2, the deionized water is mixed with the turmeric powder in the step 1 is heated to boiling, and keeping boiling for 5 minutes.

In one preferred embodiment, in the step 3, the liquid in the step 2 is cooled and then stirred under the temperature ranging 75-85° C., more preferably 85° C.

In one preferred embodiment, in the step 4, 5 ml of 1 mmol silver nitrate solution is added and stirred while carrying out the step 3.

In one preferred embodiment, in the step 5, the liquid temperature in the step 4 is lowered to 45˜55° C. and stirred for 24 hours before ending the reaction.

In one preferred embodiment, in the step 6, centrifuging is carried out at 3000 rpm under 4° C. for 10 minutes to remove the precipitate.

In one preferred embodiment, in the step 7, the supernatant is taken out of the liquid centrifugated in the step 6 from which the precipitate has been removed; in the step 8, the supernatant of the step 7 is centrifuged at 14000 rpm under 4° C. for 1 hour.

In one preferred embodiment, in the step 9, the precipitate is collected and the precipitate is crude extract of Silver-nanocurcumin; in the step 10, 50 ml of the deionized water is added to wash the crude extract of Silver-nanocurcumin with shaking.

In one preferred embodiment, in the step 11, the crude extract of Silver-nanocurcumin is centrifuged at 14000 rpm under 4° C. for 1 hour; in the step 12, the precipitate of the step 11 is collected; in the step 13, the precipitate of the step 12 is washed and centrifuged repeatedly at 14000 rpm under 4° C. to remove residual silver nitrate.

### BRIEF DESCRIPTION OF THE DRAWINGS

The present disclosure will be further described in detail below with reference to the drawings and specific embodiments.

FIG. 1 is a block diagram of the step flow of the present disclosure.

### DETAILED DESCRIPTION OF THE PREFERRED EMBODIMENTS

The technical solutions in the embodiments of the present disclosure will be clearly and completely described below in conjunction with the drawings in the embodiments of the present disclosure. In the following description, many specific details are set forth in order to fully understand the present disclosure, but the present disclosure can also be implemented in other ways different from those described here, and those skilled in the art can do so without departing from the connotation of the present disclosure. Similar promotion, therefore, the present disclosure is not limited by the specific embodiments disclosed below.

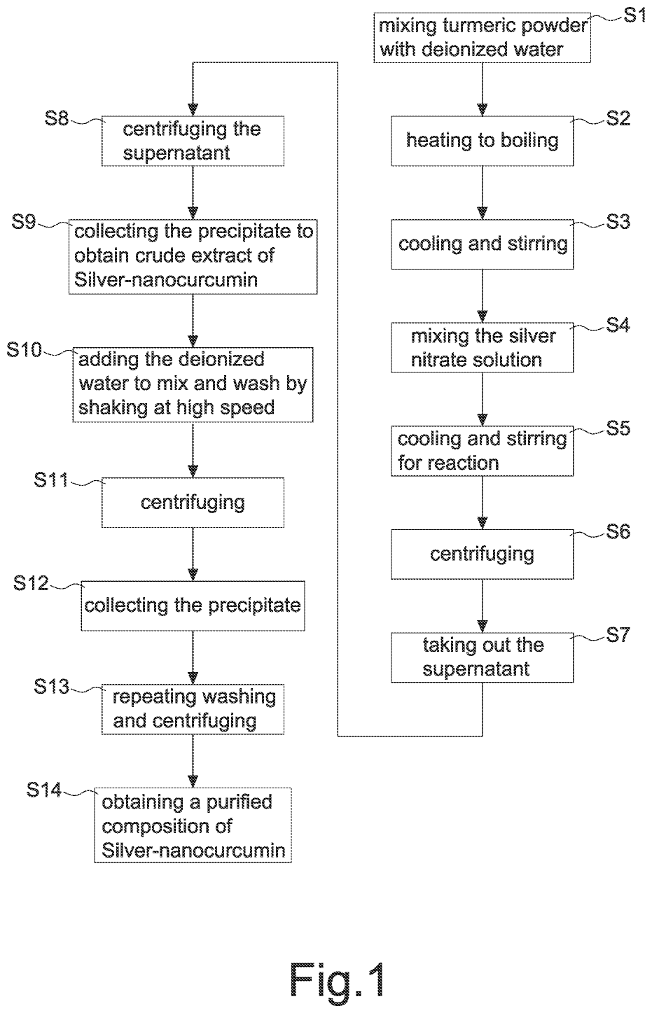

Please refer to FIG. 1, a method of preparing Silver-nanocurcumin material for inhibiting new coronavirus includes the following steps in sequence:

step1: mixing turmeric powder with deionized water;

step2: heating to boiling;

step3: cooling and stirring;

step4: mixing the silver nitrate solution;

step5: cooling and stirring for reaction;

step6: centrifuging;

step7: taking out the supernatant;

step8: centrifuging the supernatant;

step9: collecting the precipitate to obtain the crude extract of Silver-nanocurcumin;

step10: adding deionized water to mix and wash by shaking at high speed;

step11: centrifuging;

step12: collecting the precipitate;

step13: repeating washing and centrifuging; and

step14: obtaining a purified composition of Silver-nanocurcumin.

Wherein, in the step 1, 5 grams of the turmeric powder are weighed out and poured into 500 ml of the deionized water; in the step 2, the deionized water is mixed with the turmeric powder in the step 1 is heated to boiling, and keeping boiling for preferably 5 minutes; in the step 3, the liquid in the step 2 is cooled and then stirred gently under the temperature ranging 75-85° C., and preferably 85° C.; in the step 4, 5 ml of 1 mmol silver nitrate solution is added and stirred while stirring; in the step 5, the liquid temperature in the step4 is lowered to 45-55° C., preferably 50° C., and stirred gently for 24 hours then ending the reaction; in the step 6, centrifuging is carried out at 3000 rpm under 4° C. for 10 minutes to remove the precipitate; in the step 7, the supernatant is taken out of the liquid centrifugated in the step 6 from which the precipitate have been removed; in the step 8, the supernatant of the step 7 is centrifuged at 14000 rpm under 4° C. for 1 hour; in the step 9, the precipitate is collected and the precipitate is crude extract of Silver-nanocurcumin; in the step 10, 50 ml of the deionized water is added to wash the crude extract of Silver-nanocurcumin with shaking; in the step 11, the crude extract of Silver-nanocurcumin is centrifuged at 14000 rpm under 4° C. for 1 hour; in the step 12, the precipitate of the step 11 is collected; in the step 13, the precipitate of the step 12 is washed and centrifuged repeatedly at 14000 rpm under 4° C. to remove residual silver nitrat; at last, in the step 14, a purified composition of Silver-nanocurcumin is obtained.

The composition of Silver-nanocurcumin can be mixed with pure water to become a nasal spray during application, or mixed with various solids or liquids to form powder, liquid or gel-like anti-virus and anti-bacterial products.

It should be further clarified that there is no safety concern about the normal use of nano-silver in the human body. Since the human body usually consumes silver from food and water daily, the human body has a normal mechanism for processing and metabolizing silver. The silver content in most foods is in the range of 10-100 μg/kg. The biological half-life of silver in the human liver ranges from several days to 50 days, and most of the absorbed silver will be excreted with the bile in the feces.

After research, the inventors found that Silver-nanocurcumin of the present disclosure can be used to prevent the new coronavirus COVID-19 infection in healthy humans; Spraying Silver-nanocurcumin into the nasal cavity with a nasal spray can get the virus killed in the nasal cavity within a certain period of time. Spraying the appropriate amount multiple times in a day to make the nasal cavity partly with Silver-nanocurcumin within a few hours to kill the virus and block the cells to protect them from infection.

The composition of Silver-nanocurcumin prepared by the present disclosure can not only be used to inhibit the new coronavirus and its similar viruses, but also be used as a drug source for the prevention and treatment of diseases derived from the new coronavirus COVID-19 in the future. In addition, it is a biological antiviral material, which is odorless, harmless, non-toxic and non-irritating to organisms. It can be used for cleaning, disinfection, sterilization, and anti-bacterial products such as desks, masks, door handles, mobile phones, elevator keys and other items, as well as medical disinfection and wound healing, influenza virus, enterovirus and other follow-up medical research and development applications, food processing and disinfection, fresh fruit and vegetable disinfection, frozen food disinfection, cosmetic hygiene operations; also can be used in confined spaces (For example, air disinfection in airplanes, vehicles, ships, skyscrapers, and any central air-conditioning facilities) is used to reduce the COVID-19 aerosol content in the air, thereby reducing the speed and capacity of COVID-19 transmission.

Obviously, the described embodiments are only a part of the embodiments of the present disclosure, rather than all the embodiments. Based on the embodiments of the present disclosure, all other embodiments obtained by those of ordinary skill in the art without creative work shall fall within the protection scope of the present disclosure.

1. A method of preparing Silver-nanocurcumin material for inhibiting new coronavirus is characterized in that it comprises the following steps in sequence:
step1: mixing turmeric powder with deionized water;
step2: heating to boiling;
step3: cooling and stirring;
step4: mixing the silver nitrate solution;
step5: cooling and stirring for reaction;
step6: centrifuging;
step7: taking out the supernatant;
step8: centrifuging the supernatant;
step9: collecting the precipitate to obtain crude extract of Silver-nanocurcumin;
step10: adding deionized water to mix and wash by shaking at high speed;
step11: centrifuging;
step12: collecting the precipitate;
step13: repeating washing and centrifuging; and
step14: obtaining a purified composition of the Silver-nanocurcumin.

2. The method for preparing Silver-nanocurcumin material for inhibiting new coronavirus according to claim 1, characterized in that, in the step 1, 5 grams of the turmeric powder are weighed out and poured into 500 ml of the deionized water.

3. The method for preparing Silver-nanocurcumin material for inhibiting new coronavirus according to claim 2, characterized in that, in the step 2, the deionized water is mixed with the turmeric powder in the step 1 is heated to boiling, and keeping boiling for 5 minutes.

4. The method for preparing Silver-nanocurcumin material for inhibiting new coronavirus according to claim 3, characterized in that, in the step 3, the liquid in the step 2 is cooled and then stirred under the temperature ranging 75-85° C.

5. The method for preparing Silver-nanocurcumin material for inhibiting new coronavirus according to claim 4, characterized in that, in the step 4, 5 ml of 1 mmol silver nitrate solution is added and stirred while carrying out the step 3.

6. The method for preparing Silver-nanocurcumin material for inhibiting new coronavirus according to claim 5, characterized in that, in the step 5, the liquid temperature in the step 4 is lowered to 45˜55° C. and stirred for 24 hours then ending the reaction.

7. The method for preparing Silver-nanocurcumin material for inhibiting new coronavirus according to claim 6, characterized in that, in the step 6, centrifuging is carried out at 3000 rpm under 4° C. for 10 minutes to remove the precipitate.

8. The method for preparing Silver-nanocurcumin material for inhibiting new coronavirus according to claim 7, characterized in that, in the step 7, the supernatant is taken out of the liquid centrifugated in the step 6 from which the precipitate have been removed; in the step 8, the supernatant of the step 7 is centrifuged at 14000 rpm under 4° C. for 1 hour.

9. The method for preparing Silver-nanocurcumin material for inhibiting new coronavirus according to claim 8, characterized in that, in the step 9, the precipitate is collected and the precipitate is crude extract of Silver-nanocurcumin; in the step 10, 50 ml of the deionized water is added to wash the crude extract of Silver-nanocurcumin with shaking.

10. The method for preparing Silver-nanocurcumin material for inhibiting new coronavirus according to claim 9, characterized in that, in the step 11, the crude extract of Silver-nanocurcumin is centrifuged at 14000 rpm under 4° C. for 1 hour; in the step 12, the precipitate of the step 11 is collected; in the step 13, the precipitate of the step 12 is washed and centrifuged repeatedly at 14000 rpm under 4° C. to remove residual silver nitrate.
